(19) United States
(12) Patent Application Publication
(10) Pub. No.: US 2016/0009751 A1
(43) Pub. Date: Jan. 14, 2016

(54) C-19 HYDROXY PREGNENES

(71) Applicant: LEO Pharma A/S, Ballerup (DK)

(72) Inventors: Michael L. SIERRA, Ballerup (DK); Jakob FELDING, Ballerup (DK); Qianghui ZHOU, La Jolla, CA (US); Hans RENATA, Arcadia, CA (US); Philippe S. BARAN, La Jolla, CA (US)

(21) Appl. No.: 14/330,808

(22) Filed: Jul. 14, 2014

(51) Int. Cl. C07J 7/00; C07J 21/00; A61K 31/573

(52) U.S. Cl. CPC C07J 7/0085; A61K 31/573; C07J 21/008

(57) ABSTRACT

The present invention relates to a compound according to formula I

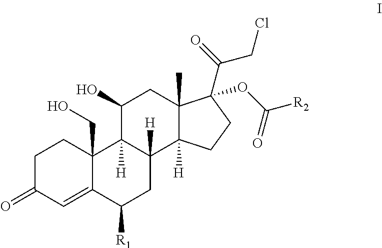

wherein R₁ is selected from the group consisting of hydrogen and fluoro and R₂ is selected from C₁-C₄ alkyl and hydrates or solvates thereof. The invention further relates to intermediates for the preparation of said compounds, to said compounds for use in therapy, to pharmaceutical compositions comprising said compounds, to methods of treating diseases with said compounds, and to the use of said compounds in the manufacture of medicaments.

### FIELD OF THE INVENTION

This invention relates to novel C-19 hydroxy pregnenes and derivatives thereof, to intermediates for the preparation thereof, to said compounds for use in therapy, to pharmaceutical compositions comprising said compounds, to methods of treating diseases with said compounds, and to the use of said compounds in the manufacture of medicaments.

### BACKGROUND OF THE INVENTION

The glucocorticoid receptor is part of the family of nuclear receptors. The receptor is a nuclear transcription factor that when bound to a ligand promotes or suppresses the transcription of genes. Glucocorticoid receptor agonists occur naturally or may be prepared synthetically.

Glucocorticoid receptor agonists are potent anti-inflammatory drugs which are used in the management of inflammatory and allergic conditions such as psoriasis, eczema, atopic dermatitis, asthma, obstructive airway diseases and rheumatoid arthritis.

Examples of marketed glucocorticoid receptor agonists include hydrocortisone and dobetasol proprionate.

N. V. Kovganko et. al. (Khimiya Prirodnykh Soedinenii, (1993), 3, 374-84) describe the preparation of 19-hydroxycortisol.

U.S. Pat. No. 3,039,926 describe 10-(1-hydroxyethyl)- and 10-(1-hydroxypropyl)-17,21-dihydroxy-19-Norpregn-4-ene-3,11,20-trione.

U.S. Pat. No. 3,245,986 describe 11β,16α,17,19,21-pentahydroxy-10α-Pregn-4-ene-3,20-dione.

U.S. Pat. No. 3,039,926 describe 19-hydroxy pregnenes.

U.S. Pat. No. 2,966,444 describe enzymatic oxygenative production of 11- or -19-position hydroxylated steroids.

There is a continuous need for developing novel glucocorticoid receptor agonists with reduced adverse effects.

### SUMMARY OF THE INVENTION

The invention provides novel C-19 hydroxy pregnene derivatives which exhibit glucocorticoid receptor agonist effect.

Compounds of the present invention may for example be beneficial in preventing, treating or ameliorating a variety of inflammatory diseases, such as psoriasis, eczema and atopic dermatitis.

Compounds of the present invention may have advantageous properties such as reduced adverse effects, for example due to favorable glucocorticoid receptor agonist effect in comparison to mineralocorticoid receptor antagonist effect.

Accordingly, the present invention relates to a compound according to formula I

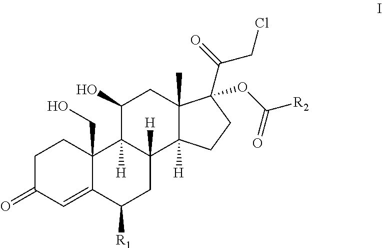

wherein
R₁ is selected from the group consisting of hydrogen and fluoro;
R₂ is selected from C₁-C₄ alkyl;
and hydrates or solvates thereof.

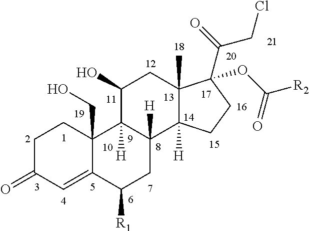

### Steroid Nomenclature

Furthermore, the invention relates to a compound according to formula I for use in therapy.

The invention furthermore relates to a pharmaceutical composition comprising a compound according to formula I together with a pharmaceutically acceptable vehicle or excipient or pharmaceutically acceptable carrier(s).

The invention also relates to intermediates for the preparation of a compound of formula I. Thus, the invention relates to a compound of general formula II

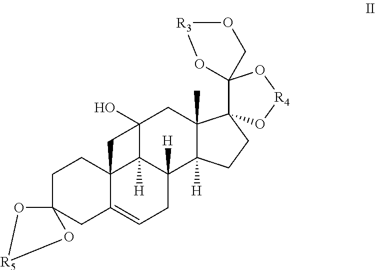

wherein R₃ and R₄ independently represent —CH₂—, —CH(CH₃)— or —C(CH₃)₂— and wherein R₅ represents —(CH₂)₂—, —(CH₂)₃— or —CH₂—C(CH₃)₂—CH—.

The invention furthermore relates to a compound of general formula III

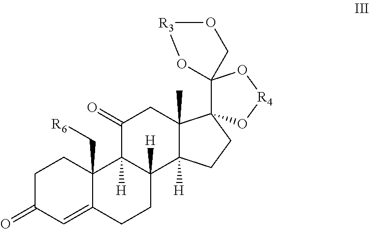

wherein R₃ and R₄ independently represent —CH₂—, —CH(CH₃)— or —C(CH₃)₂— and wherein R₆ represents halogen or hydroxyl.

### DETAILED DESCRIPTION OF THE INVENTION

### Definitions

The term “alkyl” is intended to indicate a radical obtained when one hydrogen atom is removed from a branched or linear hydrocarbon. Said alkyl comprises 1-4, such as 1-2, such as 2-3, such as 2-4 carbon atoms. The term includes the subclasses normal alkyl (n-alkyl), secondary and tertiary alkyl, such as methyl, ethyl, n-propyl, isopropyl, n-butyl, isobutyl, sec.-butyl and tert.-butyl

The term “halogen” is intended to indicate a substituent from the 7^{th} main group of the periodic table, such as fluoro, chloro, bromo and iodo.

The term “hydrocarbon radical” is intended to indicate a radical containing only hydrogen and carbon atoms, it may contain one or more double and/or triple carbon-carbon bonds, and it may comprise cyclic moieties in combination with branched or linear moieties. Said hydrocarbon comprises 1-4 carbon atoms, e.g. 1-3, e.g. 1-2, e.g. 2-3, e.g. 2-4 carbon atoms. The term includes alkyl, as indicated herein.

In some instances, the number of carbon atoms in a hydrocarbon radical (e.g. alkyl) is indicated by the prefix “(Cₐ-C_{b})”, wherein a is the minimum number and b is the maximum number of carbons in the hydrocarbon radical. Thus, for example (C₁-C₄)alkyl is intended to indicate an alkyl radical comprising from 1 to 4 carbon atoms.

The term “solvate” is intended to indicate a species formed by interaction between a compound, e.g. a compound of formula I, and a solvent, e.g. alcohol, glycerol or water, wherein said species are in a crystalline form. When water is the solvent, said species is referred to as a hydrate.

The term “treatment” as used herein means the management and care of a patient for the purpose of combating a disease, disorder or condition. The term is intended to include the delaying of the progression of the disease, disorder or condition, the amelioration, alleviation or relief of symptoms and complications, and/or the cure or elimination of the disease, disorder or condition. The term may also include prevention of the condition, wherein prevention is to be understood as the management and care of a patient for the purpose of combating the disease, condition or disorder and includes the administration of the active compounds to prevent the onset of the symptoms or complications. Nonetheless, prophylactic (preventive) and therapeutic (curative) treatments are two separate aspects.

All references, including publications, patent applications and patents, cited herein are hereby incorporated by reference.

### Embodiments of the Invention

In an embodiment the invention relates to a compound of general formula I wherein R₁ is hydrogen.

In an embodiment the invention relates to a compound of general formula I wherein R₁ is fluoro.

In an embodiment the invention relates to a compound of general formula I wherein R₂ is ethyl.

In an embodiment the invention relates to a compound of general formula I selected from (8S,9S,10S,11S,13S,14S,17R)-17-(2-chloroacetyl)-11-hydroxy-10-(hydroxymethyl)-13-methyl-3-oxo-2,3,6,7,8,9,10,11,12,13,14,15,16,17-tetradecahydro-1H-cyclopenta[a]phenanthren-17-yl propionate and (6R,8S,9S,10S,11S,13S,14S,17R)-17-(2-chloroacetyl)-6-fluoro-11-hydroxy-10-(hydroxymethyl)-13-methyl-3-oxo-2,3,6,7,8,9,10,11,12,13,14,15,16,17-tetradecahydro-1H-cyclopenta[a]phenanthren-17-yl propionate

and hydrates or solvates thereof.

In an embodiment the invention relates to a compound of general formula II which is (6a′S,6a^{1′}S,8a′S,9′R,11a′S, 11b′S)-8a′-methyl-1′,3′,5′,6′,8′,8a′,10′,11′,11a′,11 b′-decahydro-7′H-trispiro[[1,3]dioxolane-2,4′-cyclobuta[de]cyclopenta[a]phenanthrene-9′,4″-[1,3]dioxolane-5″,4′″-[1,3]dioxolan]-7a′(6a^{1′}H)-ol.

In an embodiment the invention relates to a compound of general formula III which is (8S,9S,10S,13S,14S,17R)-10-(hydroxymethyl)-13-methyl-1,2,6,7,8,9,10,12,13,14,15,16-dodecahydrodispiro[cyclopenta[a]phenanthrene-17,4′-[1,3]dioxolane-5′,4″-[1,3]dioxolane]-3,11-dione.

The compounds of formula I may be obtained in crystalline form either directly by concentration from an organic solvent or by crystallisation or recrystallisation from an organic solvent or mixture of said solvent and a cosolvent that may be organic or inorganic, such as water. The crystals may be isolated in essentially solvent-free form or as a solvate, such as a hydrate. The invention covers all crystalline forms, such as polymorphs and pseudopolymorphs, and also mixtures thereof.

Furthermore, when a double bond or a fully or partially saturated ring system is present in the molecule geometric isomers may be formed. It is intended that any geometric isomer, as separated, pure or partially purified geometric isomers or mixtures thereof are included within the scope of the invention.

In one or more embodiments of the present invention, the compounds of formula I as defined above are useful in therapy and in particular useful for treatment of inflammatory diseases, such as psoriasis, eczema and atopic dermatitis.

Compounds of the present invention, optionally in combination with other active compounds, would be useful for the treatment of inflammatory diseases or conditions, in particular for the treatment of psoriasis, eczema and atopic dermatitis.

In one or more embodiments of the present invention, the compounds of formula I as defined above are useful in the manufacture of a medicament for the prophylaxis, treatment or amelioration of inflammatory diseases.

In one or more embodiments of the present invention, the compounds of formula I as defined above are useful in the manufacture of a medicament for the prophylaxis, treatment or amelioration of psoriasis, eczema or atopic dermatitis.

In one or more embodiments the present invention relates to methods of preventing, treating or ameliorating inflammatory diseases, the method comprising administering to a person suffering from at least one of said diseases an effective amount of one or more compounds according to according to the compounds of formula I, optionally together with a pharmaceutically acceptable carrier or one or more excipients, optionally in combination with other therapeutically active compounds.

In one or more embodiments the present invention relates to methods of preventing, treating or psoriasis, eczema and atopic dermatitis, the method comprising administering to a person suffering from at least one of said diseases an effective amount of one or more compounds according to according to the compounds of formula I, optionally together with a pharmaceutically acceptable carrier or one or more excipients, optionally in combination with other therapeutically active compounds.

Besides being useful for human treatment, the compounds of the present invention may also be useful for veterinary treatment of animals including mammals such as horses, cattle, sheep, pigs, dogs, and cats.

### Pharmaceutical Compositions of the Invention

For use in therapy, compounds of the present invention are typically in the form of a pharmaceutical composition. The invention therefore relates to a pharmaceutical composition comprising a compound of formula I, optionally together with one or more other therapeutically active compound(s), together with a pharmaceutically acceptable excipient, vehicle or carrier(s). The excipient must be “acceptable” in the sense of being compatible with the other ingredients of the composition and not deleterious to the recipient thereof.

Conveniently, the active ingredient comprises from 0.0001-99.9% by weight of the formulation.

In the form of a dosage unit, the compound may be administered one or more times a day at appropriate intervals, always depending, however, on the condition of the patient, and in accordance with the prescription made by the medical practitioner. Conveniently, a dosage unit of a formulation contain between 0.001 mg and 1000 mg, preferably between 0.01 mg and 100 mg, such as 0.1-50 mg of a compound of formula I.

A suitable dosage of the compound of the invention will depend, inter alia, on the age and condition of the patient, the severity of the disease to be treated and other factors well known to the practising physician. The compound may be administered either orally, parenterally, topically, transdermally or interdermally according to different dosing schedules, e.g. daily, weekly or with monthly intervals. In general a single dose will be in the range from 0.001 to 400 mg/kg body weight. The compound may be administered as a bolus (i.e. the entire daily doseis is administered at once) or in divided doses two or more times a day.

In the context of topical treatment it may be more appropriate to refer to a “usage unit”, which denotes a single dose which is capable of being administered to a patient, and which may be readily handled and packed, remaining as a physically and chemically stable unit dose comprising either the active material as such or a mixture of it with solid, semisolid or liquid pharmaceutical diluents or carriers.

The term “usage unit” in connection with topical use means a unitary, i.e. a single dose, capable of being administered topically to a patient in an application per square centimetre of the treatment area of from 0.001 microgram to 1 mg and preferably from 0.05 microgram to 0.5 mg of the active ingredient in question.

It is also envisaged that in certain treatment regimes, administration with longer intervals, e.g. every other day, every week, or even with longer intervals may be beneficial.

If the treatment involves administration of another therapeutically active compound it is recommended to consult Goodman & Gilman's The Pharmacological Basis of Therapeutics, 9^{th} Ed., E. G. Hardman and L. E. Limbird (Eds.), McGraw-Hill 1995, for useful dosages of said compounds.

The administration of a compound of the present invention with one or more other active compounds may be either concomitantly or sequentially.

The formulations include e.g. those in a form suitable for oral (including sustained or controlled release), parenteral (including subcutaneous, intraperitoneal, intramuscular, intraarticular and intravenous), transdermal, intradermal or topical administration.

The formulations may conveniently be presented in dosage unit form and may be prepared by but not restricted to any of the methods well known in the art of pharmacy, e.g. as disclosed in Remington, The Science and Practice of Pharmacy, 21ed ed., 2005.

All methods include the step of bringing the active ingredient into association with the carrier, which constitutes one or more accessory ingredients. In general, the formulations are prepared by uniformly and intimately bringing the active ingredient into association with a liquid carrier, semisolid carrier or a finely divided solid carrier or combinations of these, and then, if necessary, shaping the product into the desired formulation.

Formulations of the present invention suitable for oral administration may be in the form of discrete units as capsules, sachets, tablets, chewing gum or lozenges, each containing a predetermined amount of the active ingredient; in the form of a powder, granules or pellets; in the form of a solution or a suspension in an aqueous liquid or non-aqueous liquid, such as ethanol or glycerol; or in the form of a gel, a nano- or microemulsion, an oil-in-water emulsion, a water-in-oil emulsion or other dispensing systems. The oils may be edible oils, such as but not restricted to e.g. cottonseed oil, sesame oil, coconut oil or peanut oil. Suitable dispersing or suspending agents for aqueous suspensions include synthetic or natural surfactants and viscosifyring agents such as but not restricted to tragacanth, alginate, acacia, dextran, sodium carboxymethylcellulose, gelatin, methylcellulose, hydroxypropylmethylcellulose, hydroxypropylcellulose, carbomers, polyvinylpyrrolidone, polysorbates, sorbitan fatty acid esters. The active ingredients may also be administered in the form of a bolus, electuary or paste.

A tablet may be made by compressing, moulding or freeze drying the active ingredient optionally with one or more accessory ingredients. Compressed tablets may be prepared by compressing, in a suitable machine, the active ingredient(s) in a free-flowing form such as a powder or granules, optionally mixed by a binder and/or filler, such as e.g. lactose, glucose, mannitol starch, gelatine, acacia gum, tragacanth gum, sodium alginate, calcium phosphates, microcrystalline cellulose, carboxymethylcellulose, methylcellulose, hydroxypropylcellulose, hydroxypropylmethylcellulose, ethylcellulose, hydroxyethyicellulose, polyethylene glycol, waxes or the like; a lubricant such as e.g. sodium oleate, sodium stearate, magnesium stearate, sodium benzoate, sodium acetate, sodium chloride or the like; a disintegrating agent such as e.g. starch, methylcellulose, agar, bentonite, croscarmellose sodium, sodium starch glycollate, crospovidone or the like or a dispersing agent, such as polysorbate 80. Moulded tablets may be made by moulding, in a suitable machine, a mixture of the powdered active ingredient and suitable carrier moistened with an inert liquid diluent. Freeze dryed tablets may be formed in a freeze-dryer from a solution of the drug substance. A suitable filler can be included.

Formulations suitable for parenteral administration conveniently comprise a sterile oily or aqueous preparation of the active ingredients, which is preferably isotonic with the blood of the recipient, e.g. isotonic saline, isotonic glucose solution or buffer solution. Furthermore, the formulation may contain cosolvent, solubilising agent and/or complexation agents. The formulation may be conveniently sterilised by for instance filtration through a bacteria retaining filter, addition of sterilising agent to the formulation, irradiation of the formulation or heating of the formulation. Liposomal formulations as disclosed in e.g. Encyclopedia of Pharmaceutical Technology, vol. 9, 1994, are also suitable for parenteral administration.

Alternatively, the compounds of formula I may be presented as a sterile, solid preparation, e.g. a freeze-dried powder, which is readily dissolved in a sterile solvent immediately prior to use.

Transdermal formulations may be in the form of a plaster, patch, microneedles, liposomal or nanoparticulate delivery systems or other cutaneous formulations applied to the skin.

Formulations suitable for topical, such as dermal or intradermal administration include liquid or semi-solid preparations such as liniments, lotions, gels, applicants, sprays, foams, filmforming systems, microneedles, micro- or nano-emulsions, oil-in-water or water-in-oil emulsions such as creams, ointments or pastes; or solutions or suspensions such as drops.

For topical administration, the compound of formula I may typically be present in an amount of from 0.001 to 20% by weight of the composition, such as 0.01% to about 10%, but may also be present in an amount of up to about 100% of the composition.

In addition to the aforementioned ingredients, the formulations of a compound of formula I may include one or more additional ingredients such as diluents, buffers, flavouring agents, colourant, surface active agents, thickeners, penetration enhancing agents, solubility enhancing agents preservatives, e.g. methyl hydroxybenzoate (including anti-oxidants), emulsifying agents and the like.

The pharmaceutical composition may additionally comprise one or more other active components conventionally used in the treatment of inflammatory diseases such as psoriasis, eczema and atopic dermatitis.

### Methods of Preparation

The compounds of the present invention can be prepared in a number of ways well known to those skilled in the art of synthesis. The compounds of formula I may for example be prepared using the reactions and techniques outlined below together with methods known in the art of synthetic organic chemistry, or variations thereof as appreciated by those skilled in the art. Preferred methods include, but are not limited to, those described below. The reactions are carried out in solvents appropriate to the reagents and materials employed and suitable for the transformations being effected. Also, in the synthetic methods described below, it is to be understood that all proposed reaction conditions, including choice of solvent, reaction atmosphere, reaction temperature, duration of experiment and work-up procedures, are chosen to be conditions of standard for that reaction, which should be readily recognized by one skilled in the art. Not all compounds falling into a given class may be compatible with some of the reaction conditions required in some of the methods described. Such restrictions to the substituents which are compatible with the reaction conditions will be readily apparent to one skilled in the art and alternative methods can be used. The compounds of the present invention or any intermediate may be purified if required using standard methods well known to a synthetic organist chemist, e.g. methods described in “Purification of Laboratory Chemicals”, 6^{th} ed. 2009, W. Amarego and C. Chai, Butterworth-Heinemann. Starting materials are either known compounds, commercially available, or they may be prepared by routine synthetic methods well known to a person skilled in the art.

### General Procedures, Preparations and Examples

¹H nuclear magnetic resonance (NMR) spectra were recorded at 400 MHz unless otherwise specified. Chemical shift values (δ, in ppm) are quoted relative to internal tetramethylsilane (δ=0.00) standards. The value of a multiplet, either defined doublet (d), triplet (t), quartet (q)) or not (m) at the approximate midpoint is given unless a range is quoted. (br) indicates a broad peak, whilst (s) indicates a singlet.

The following abbreviations have been used throughout:

DCE 1,2-dichloroethane
DCM dichloromethane
DMAP 4-dimethylaminopyridine

### DMF N,N-Dimethylformamide

DMSO dimethyl sulfoxide
Et ethyl
EtOAc ethyl acetate
h hour(s)
HPLC High pressure liquid chromatography
HRMS High resolution mass spectroscopy
IR Infrared spectroscopy
L litre
m milli
Me methyl
MS Mass spectrometry

### NIS N-iodosuccmnimide

NMR nuclear magnetic resonance
PTLC preparative thin-layer chromatography
rt room temperature
RT Retention lime
Selectfluor 1-Chloromethyl-4-fluoro-1,4-diazoniabicyclo[2.2.2]octane bis(tetrafluoroborate)
TFA trifluoroacetic acid
TMSCl Timethylsilyl chloride
TMSOTf Trimethylsilyl trifluoromethanesulfonate
THF tetrahydrofuran
v volume

### PREPARATIONS AND EXAMPLES

### Intermediate 2

### (6a′S,6a^{1′}S,8a′S,9′R, 11a′S,11 b′S)-8a′-methyl-1′,3′,5′,6′,8′8a′, 10′,11′,11a′,11b′-decahydro-7′H-trispiro[[1,3]dioxolane-2,4′-cyclobuta[de]cyclopenta[a]phenanthrene-9′,4″-[1,3]dioxolane-5″,4′″-[1,3]dioxolan]-7a′(6a^{1′}H-ol

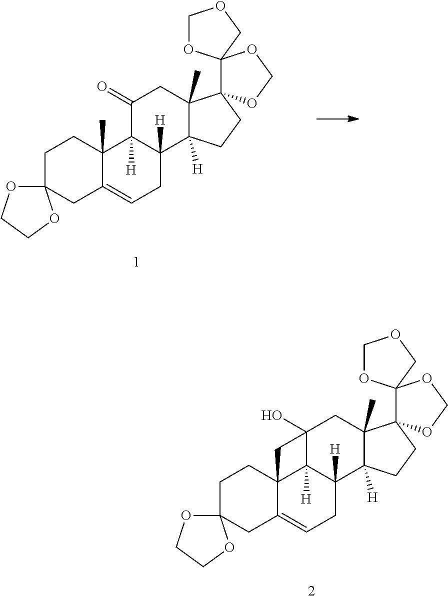

Triketal 1 ((8′S,9′S, 10′R, 13′S, 14′S, 17′R)-10′,13′-dimethyl-1′,2′,4′,7′,8′,9′,10′,12′,13′,14′,15′,16′-dodecahydro-11′H-trispiro[[1,3]dioxolane-2,3′-cyclopenta[a]phenanthrene-17′,4″-[1,3]dioxolane-5″,4′″-[1,3]dioxolan]-11′-one)(3.00 g, 6.72 mmol) (Fried, J. H.; Arth, G. E.; Sarett, L. H. J. Am. Chem. Soc. (1959), 81, 1235) was dissolved in dry ethanol (1.0 L), transferred to a photoreactor [ACE glass, 1 L jacketed reaction vessel with quartz immersion well], and degassed with argon for 15 minutes, followed by irradiation using a 450 W mercury Hanovia lamp through a vycor filter for 60 h. The solution was then poured into a roundbottom flask and concentrated in vacuo. The resulting oil was purified by column chromatography (1:4 to 2:3 EtOAc:hexanes) to give intermediate 2 (1.71 g, 57% yield).

Physical state: white foam;

R_{f}=0.27 (silica gel, 2:3 EtOAc:hexanes);

[α]^{D} ,25=−51.0° (c=0.48, CHCl₃);

HRMS (m/z): calcd for C₂₅H₃₄O₇, [M+H]⁺, 447.2377. found, 447.2380.

IR (film) λₘₐₓ: 3439, 2941, 2881, 1707, 1438, 1324, 1240, 1215, 1180, 1100, 1081;

¹H NMR (400 MHz, CDCl₃): δ 5.65 (d, J=6.0 Hz, 1H), 5.21 (s, 1H), 5.08 (d, J=1.1 Hz, 1H), 5.06 (d, J=1.1 Hz, 1H), 5.01 (s, 1H), 4.01-3.93 (m, 6H), 2.87 (dt, J=13.3, 3.4 Hz, 1H), 2.49 (d, J=11.7 Hz, 1H), 2.22-2.17 (m, 2H), 2.10 (dd, J=11.7, 4.9 Hz, 1H), 2.05-1.92 (m, 3H), 1.88-1.68 (m, 9H), 1.53-1.34 (m, 2H), 0.97 (s, 3H);

¹³C NMR (CDCl₃, 151 MHz): δ 141.6, 126.0, 109.9, 109.7, 95.0, 91.9, 91.8, 77.4, 70.2, 64.6, 64.5, 57.0, 50.1, 45.8, 45.5, 43.6, 42.5, 41.8, 37.5, 34.7, 32.2, 32.1, 27.6, 21.7, 15.6.

### Intermediate 3

### (8′S,9′S,10′S,13′S,14′S,17′R)-10′-(iodomethyl)-13′-methyl-1′,2′,4′,7′,8′,9′,10′,12′,13′,14′,15′,16′-dodecahydro-11′H-trispiro[[1,3]dioxolane-2,3′-cyclopenta[a]phenanthrene-17′,4″-[1,3]dioxolane-5″,4′″-[1,3]dioxolan]-11′-one

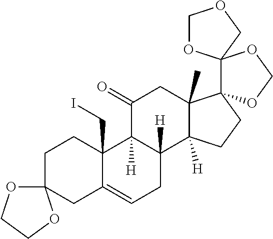

The solution of intermediate 2 (0.283 g, 0.63 mmol) in toluene (12 mL) was degassed with Ar for 15 min, followed by sequential addition of MeOH (0.257 mL, 6.34 mmol, 10.0 equiv), Li₂CO₃ (164 mg, 2.22 mmol, 3.5 equiv) and NIS (428 mg, 1.90 mmol, 3.0 equiv) under Ar. The resulting suspension was irradiated with sunlamp (90 W, 6 inches from the walls of the flask) for 15 minutes. Irradiation was then halted and the crude mixture was washed with saturated aqueous Na₂S₂O₃ (15 mL) until colorless². The aqueous layer was back-extracted with EtOAc (15 mL) and the organic layer was washed with brine (15 mL), dried over Na₂SO₄ and concentrated in vacuo. The resulting yellow oil was used for the next step without further purification. This yellow oil could also be purified by silica gel chromatography (1:3 EtOAc:hexanes) to give intermediate 3.

Physical state: white foam;

R_{f}=0.65 (silica gel, 1:1 EtOAc:hexanes);

[α]^{D},25=−61.5° (c=1.0, CH₂Cl₂);

HRMS (m/z): calcd for C₂₅H₃₃IO₇, [M+H]⁺, 573.1344. found, 573.1317.

IR (film) λₘₐₓ: 2944, 2877, 1696, 1095, 941, 732;

¹H NMR (400 MHz, CDCl₃): δ 5.65-5.58 (m, 1H), 5.20 (s, 1H), 5.07 (d, J=1.0 Hz, 1H), 5.05 (d, J=1.0 Hz, 1H), 5.02 (s, 1H), 4.35 (d, J=11.0 Hz, 1H), 4.02-3.87 (m, 6H), 3.63 (dd, J=11.0, 1.3 Hz, 1H), 2.92-2.82 (m, 1H), 2.83 (dd, J=13.5, 1.2 Hz, 1H), 2.66 (d, J=14.4 Hz, 1H), 2.66-2.61 (m, 1H), 2.41 (dt, J=14.3, 2.7 Hz, 1H), 2.27-2.07 (m, 3H), 2.04 (d, J=11.8 Hz, 1H), 1.99-1.72 (m, 5H), 1.63 (ddt, J=15.2, 5.6, 3.4 Hz, 1H), 1.52-1.32 (m, 2H), 0.95 (s, 3H);

¹³C NMR (CDCl₃, 151 MHz): δ 212.2, 137.9, 124.0, 109.7, 108.5, 95.2, 91.9, 91.1, 70.0, 64.6, 64.5, 60.6, 51.5, 51.0, 48.9, 41.6, 39.6, 35.7, 32.7, 32.1, 31.9, 31.1, 23.6, 14.8, 10.1.

### Intermediate 4

### (8S,9S,10S,13S,14S,17R)-10-(iodomethyl-13-methyl-1,2,6,7,8,9,10,12,13,14,15,16-dodecahydrodispiro[cyclopenta[a]phenanthrene-17,4′-[1,3]dioxolane-5′,4″-[1,3]dioxolane]-3,11-dione

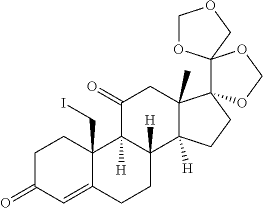

Crude intermediate 3 (0.63 mmol) from previous step was dissolved in DCM (1.0 mL), and the solution was cooled to 0° C., followed by addition of TFA (0.2 mL) and H₂O (0.1 mL). The mixture was stirred at the same temperature for 2 h. Upon completion, the reaction was quenched with saturated NaHCO₃ solution, and extracted with DCM (2×5 mL). The combined organic phases were washed with brine, dried over Na₂SO₄, and concentrated in vacuo. The resulting oil was purified by column chromatography (1:2 to 1:1 EtOAc:hexanes) to give intermediate 4 (223 mg, 67% yield for 2 steps).

Physical state: white foam;

R_{f}=0.45 (silica gel, 1:1 EtOAc:hexanes);

[α]^{D} ,25=+31.7° (c=0.30, CH₂Cl₂);

HRMS (m/z): calcd for C₂₃H₃₀IO₆, [M+H]⁺, 529.1082. found, 529.1082.

IR (film) λₘₐₓ: 2938, 2876, 1702, 1670, 1275, 1098, 944, 750;

¹H NMR (400 MHz, CDCl₃): δ 5.96 (s, 1H), 5.20 (s, 1H), 5.07 (d, J=1.0 Hz, 1H), 5.04 (d, J=1.0 Hz, 1H), 5.00 (s, 1H), 4.16 (d, J=10.4 Hz, 1H), 4.01-3.90 (m, 2H), 3.52 (d, J=10.4 Hz, 1H), 2.87 (d, J=12.9 Hz, 1H), 2.74 (ddd, J=16.7, 9.0, 5.4 Hz, 1H), 2.65-2.57 (m, 1H), 2.57 (d, J=12.8 Hz, 1H), 2.44 (dd, J=11.6, 1.0 Hz, 1H), 2.39-2.22 (m, 4H), 2.15 (ddd, J=14.5, 9.0, 5.4 Hz, 1H), 2.06 (dt, J=11.9, 3.8 Hz, 1H), 2.00-1.75 (m, 4H), 1.42 (ddd, J=17.5, 11.9, 6.1 Hz, 1H), 1.34-1.19 (m, 1H), 0.81 (d, J=0.9 Hz, 3H);

¹³C NMR (CDCl₃, 151 MHz): δ 210.5, 198.9, 165.4, 127.1, 109.5, 95.1, 91.9, 90.56, 70.0, 61.7, 50.8, 50.6, 49.9, 41.7, 36.7, 34.3, 33.4, 32.8, 32.1, 31.9, 23.1, 13.8, 12.2.

### Intermediate 5

### (8S,9S,10S,13S,14S,17R)-10-(hydroxymethyl)-13-methyl-1,2,6,7,8,9,10,12,13,14,15,16-dodecahydrodispiro[cyclopenta[a]phenanthrene-17,4′-[1,3]dioxolane-5′,4″-[1,3]dioxolane]-3,11-dione

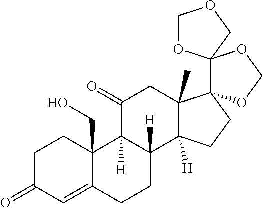

Intermediate 4 (140 mg, 0.265 mmol, 1.0 equiv) was dissolved in MeCN (6 mL), followed by sequential addition of H₂O (0.6 mL) and AgF (127 mg, 1.0 mmol, 3.8 equiv). The resulting mixture was stirred at room temperature overnight. Upon completion, the reaction mixture was diluted with EtOAc (10 mL), filtered through a pad of celite and concentrated in vacuo. The resulting yellow solid was purified by silica gel chromatography (40:1 DCM:MeOH) to give intermediate 5 (109 mg, 98%).

Physical state: white solid (m.p. 174° C.);

R_{f}=0.21 (silica gel, 3:2 EtOAc:hexanes);

[α]^{D} ,25=+35.7° (c=0.83, CHCl₃);

HRMS (m/z): calcd for C₂₃H₃₀O₇, [M+H]⁺, 419.2064. found, 419.2062.

IR (film) λₘₐₓ: 3422, 2940, 2877, 1701, 1661, 1455, 1436, 1347, 1219, 1097, 1033, 942;

¹H NMR (400 MHz, CDCl₃): δ 5.88-5.73 (m, 1H), 5.21 (s, 1H), 5.07 (dd, J=9.6, 1.0 Hz, 2H), 5.01 (s, 1H), 4.17 (d, J=11.7 Hz, 1H), 4.02-3.93 (dd, J=13.9, 9.5 Hz, 2H), 3.75 (d, J=11.6 Hz, 1H), 2.96 (d, J=12.5 Hz, 1H), 2.82 (ddd, J=13.6, 4.9, 3.6 Hz, 1H), 2.70-2.60 (m, 1H), 2.64 (d, J=12.5 Hz, 1H), 2.39-2.22 (m, 5H), 2.05-1.78 (m, 5H), 1.66-1.55 (m, 2H), 1.49-1.24 (m, 2H), 0.84 (s, 3H).

¹³C NMR (CDCl₃, 151 MHz): δ 215.1, 199.7, 165.4, 125.6, 109.6, 95.1, 92.0, 90.6, 70.0, 64.4, 64.2, 51.1, 50.8, 50.3, 43.4, 37.3, 34.4, 33.0, 32.2, 32.2, 31.4, 23.1, 13.7.

### Intermediate 6

### (8S,9S,10S,11S,13S,14S,17R)-11-hydroxy-10-(hydroxymethyl)-13-methyl-1,6,7,8,9,10,11,12,13,14,15,16-dodecahydrodispiro[cyclopenta[a]phenanthrene-17,4′-[1,3]dioxolane-5′,4″-[1,3]dioxolan]-3(2H)-one

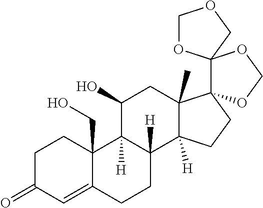

To a solution of intermediate 5 (666 mg, 1.59 mmol) in 1:1 DCM:EtOH (15 mL) at 0° C. was added NaBH₄ (19 mg, 0.5 mmol, 0.3 eq). Two more portions of NaBH₄ (19 mg each, 0.5 mmol, 0.3 eq) were added in 40 min intervals. Upon completion of the reaction as judged by TLC analysis (approximately 2 hours), any excess reducing agent was quenched by addition of 0.1 mL of wet acetone. The reaction mixture was diluted with EtOAc (50 mL) and washed sequentially with H₂O (25 mL), and brine (25 mL), dried over Na₂SO₄ and concentrated in vacuo. The resulting crude mixture was purified by column chromatography (1:3 to 1:1 EtOAc:hexanes) to afford intermediate 6 (480 mg, 72%) (Kovzanko, N. V.; Kashkan, Zh. N.; Yu. G. Chemov. Chem. Nat. Compd. (1993), 29, 374).

Physical state: white foam;

R_{f}=0.40 (silica gel, 2:1 EtOAc:hexanes);

[α]_{D},25=+6.4° (c=0.58, CHCl₃);

HRMS (m/z): calcd for C₂₃H₃₃O₇, [M+H]⁺, 421.2221. found, 421.2219.

IR (film) λₘₐₓ: 2929, 2875, 1707, 1658, 1359, 1222, 1084, 941;

¹H NMR (600 MHz, CDCl₃): δ 5.76 (s, 1H), 5.21 (s, 1H), 5.03 (s, 1H), 5.03 (dd, J=2.7, 1.0 Hz, 2H), 4.42 (q, J=3.0 Hz, 1H), 4.12 (d, J=11.9 Hz, 1H), 4.04-3.96 (m, 2H), 3.56 (d, J=11.8 Hz, 1H), 2.59 (dt, J=13.2, 4.5 Hz, 1H), 2.49 (ddd, J=16.9, 13.7, 4.8 Hz, 1H), 2.34 (dt, J=17.0, 4.2 Hz, 1H), 2.29 (dtd, J=11.0, 5.2, 3.2 Hz, 2H), 2.16 (dd, J=15.1, 3.2 Hz, 1H), 2.14-2.05 (m, 2H), 2.04-1.98 (m, 1H), 1.87-1.80 (m, 1H), 1.80-1.60 (m, 4H), 1.43 (tdd, J=12.3, 10.5, 6.9 Hz, 1H), 1.19 (dd, J=11.3, 2.8 Hz, 1H), 1.15-1-10 (m, 1H), 1.13 (s, 3H);

¹³C NMR (CDCl₃, 151 MHz): δ13C NMR (151 MHz, D2O) δ 199.5, 169.3, 123.7, 110.0, 95.0, 91.6 (2C), 70.4, 67.9, 63.8, 56.6, 53.2, 45.8, 44.3, 39.9, 34.0, 33.1, 32.2, 31.5, 31.1, 29.9, 23.7, 15.7.

### Intermediate 7

### (4aS,4a¹S,8aS,9aS,10R,12aS,12bS)-10-hydroxy-10-(2-hydroxyacetyl)-9a-methyl-3,4,8a,9,9a,10,11,12,12a,12b,13,14-dodecahydro-5H-cyclopenta[1,2]phenanthro[4,4b-de][1,3]dioxepin-2(4a¹H)-one

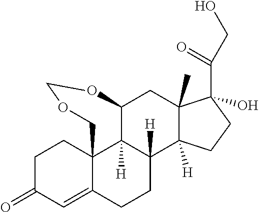

Intermediate 6 (480 mg, 1.14 mmol) was dissolved in TFA (10 mL), followed by the addition of 6 M HCl (10 mL) solution at room temperature. After 30 min, the mixture was diluted with EtOAc (25 mL) and H₂O (10 mL), cooled to 0° C., and quenched by slow addition of solid NaHCO₃ (400 mg). The layers were separated, and the organic phase was further washed with sat. aq. NaHCO₃ (25 mL). The aqueous phase was then back-extracted with EtOAc (2×25 mL), and the combined organic portions were washed with brine (25 mL), dried over Na₂SO₄, and concentrated in vacuo. The crude mixture was purified by column chromatography (2:3 to 3:2 EtOAc:hexanes) to afford intermediate 7 (235 mg, 53%).

Physical state: white solid (decomp. 157° C.);

R_{f}=0.26 (silica gel, 3:2 EtOAc:hexanes);

[α]^{D} ,25=+99.1° (c=0.57, CHCl₃);

HRMS (m/z): calcd for C₂₂H₃₀O₆, [M+H]⁺, 391.2115. found, 391.2120.

IR (film) λₘₐₓ: 3408, 2935, 1706, 1655, 1129, 1073, 1048, 911, 729;

¹H NMR (400 MHz, CDCl₃): δ. 5.84 (s, 1H), 4.70 (dd, J=23.0, 4.1 Hz, 2H), 4.67 (dd, J=19.8, 4.6 Hz, 1H), 4.29 (dd, J=19.8, 4.3 Hz, 1H), 4.16 (s, 1H), 3.96 (d, J=12.1 Hz, 1H), 3.55 (d, J=12.1 Hz, 1H), 3.06 (t, J=4.9 Hz, 1H), 2.81-2.70 (m, 1H), 2.70-2.60 (m, 1H), 2.53 (ddd, J=19.2, 14.5, 4.8 Hz, 1H), 2.44-2.30 (m, 2H), 2.20 (t, J=13.2 Hz, 1H), 2.09-1.99 (m, 1H), 2.03-1.76 (m, 4H), 1.70 (dd, J=13.5, 3.0 Hz, 1H), 1.51-1.47 (m, 2H), 1.15 (dd, J=13.1, 3.4 Hz, 1H), 1.09 (d, J=10.5 Hz, 1H), 0.93 (s, 3H).

¹³C NMR (CDCl₃, 151 MHz): δ 212.0, 199.4, 166.6, 126.6, 92.9, 88.7, 73.7, 67.4, 64.6, 57.0, 51.6, 49.0, 43.6, 36.6, 34.5, 34.0, 33.7, 32.7, 32.2, 30.4, 23.4, 18.0.

### Intermediate 8

### (4aS,4a¹S,8aS,9aS,10R,12aS,12bS)-10-(2-chloroacetyl)-10-hydroxy-9a-methyl-3,4,8a,9,9a,10,11,12,12a,12b,13,14-dodecahydro-5H-cyclopenta[1,2]phenanthro[4,4b-de][1,3]dioxepin-2(4a¹H)-one

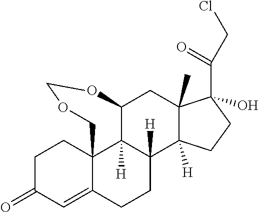

To a solution of intermediate 7 (235 mg, 0.602 mmol, 1.0 equiv) in DCM (6 mL) was added Et₃N (168 μL, 1.205 mmol, 2.0 equiv), MsCl (61 μL, 0.788 mmol, 1.3 equiv) and DMAP (7.4 mg, 0.0602 mmol, 0.1 equiv) at 0° C. After 10 min, the resulting yellow solution was warmed to room temperature and further stirred for 30 min, upon which TLC analysis showed that intermediate 7 had been fully consumed. The mixture was quenched with the addition of MeOH (100 μL) and concentrated in vacuo. The resulting yellow oil was then redissolved in DMF (6 mL) and LiCl (33.4 mg, 0.788 mmol, 1.3 equiv) was added. The mixture was heated at 60° C. for 3 h, and then diluted with EtOAc (15 mL) and washed with H₂O (15 mL). The aqueous phase was extracted with EtOAc (2×25 mL), and the combined organic portions were washed with brine (25 mL), dried with Na₂SO₄, and concentrated in vacuo. The crude product was purified by column chromatography (2:3 to 1:1 EtOAc:hexanes) to afford intermediate 8 (200 mg, 81%).

Physical state: white solid (decomp. 206° C.);

R_{f}=0.20 (silica gel, 2:3 EtOAc:hexanes);

[α]^{D},25=+138.3° (c=0.70, CHCl₃);

HRMS (m/z): calcd for C₂₂H₃₀ClO₅, [M+H]⁺, 409.1776. found, 409.1780.

IR (film) λₘₐₓ: 3442, 2928, 2853, 1726, 1655, 1611, 1459, 1368, 1261, 1233, 1127, 1077, 1047;

¹H NMR (400 MHz, CDCl₃): δ 5.84 (s, 1H), 4.73 (d, J=4.2 Hz, 1H), 4.68 (d, J=4.1 Hz, 1H), 4.62 (d, J=16.5 Hz, 1H), 4.28 (d, J=16.5 Hz, 1H), 4.16 (q, J=2.9 Hz, 1H), 3.96 (d, J=12.2 Hz, 1H), 3.55 (dd, J=12.2, 1.5 Hz, 1H), 2.93-2.81 (m, 1H), 2.66 (ddd, J=13.0, 4.9, 2.6 Hz, 1H), 2.53 (ddd, J=17.6, 14.6, 4.9 Hz, 1H), 2.43-2.31 (m, 2H), 2.20 (tdd, J=14.4, 4.4, 2.1 Hz, 2H), 2.08-2.00 (m, 2H), 1.96-1.88 (m, 1H), 1.86-1.77 (m, 2H), 1.73 (dd, J=13.3, 3.0 Hz, 1H), 1.65-1.44 (m, 2H), 1.20-1.06 (m, 2H), 0.92 (s, 3H).

¹³C NMR (CDCl₃, 151 MHz): 5202.9, 199.4, 166.6, 126.6, 92.9, 90.1, 73.6, 64.6, 56.9, 51.7, 48.6, 47.8, 43.6, 36.8, 35.0, 34.0, 33.7, 32.6, 32.2, 30.4, 29.9, 23.3, 18.0.

### Intermediate 9

### (4aS,4a′S,8aS,9aS,10R,12aS,12bS)-10-(2-chloroacetyl)-9a-methyl-2-oxo-2,3,4,4a¹,8a,9,9a, 10,11,12,12a,12b,13,14-tetradecahydro-5H-cyclopenta[1,2]phenanthro[4,4b-de][1,3]dioxepin-10-yl propionate

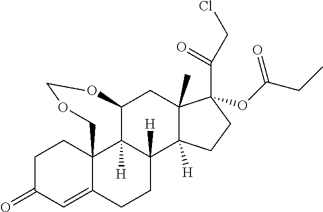

To a solution of intermediate 8 (4.0 mg, 9.8 μmmol, 1.0 equiv) in DCM (0.2 mL), was added propionic anhydride (2 μL, 14.7 μmmol, 1.5 equiv) and TMSOTf (0.75 μL, 4.4 μmmol, 0.45 equiv) successively at 0° C. under Argon. The mixture was stirred for 1 h at the same temperature, then quenched with Et₃N, and concentrated under vacuo. The resulting residue was purified by PTLC (silica gel, 1:1 EtOAc:hexanes) to afford intermediate 9 (4.5 mg, 74%).

Physical state: white foam;

R_{f}=0.65 (silica gel, 1:1 EtOAc:hexanes);

[α]_{D},25=+77.5° (c=0.28, CHCl₃);

HRMS (m/z): calcd for C₂₅H₃₄ClO₆, [M+H]⁺, 465.2038. found, 465.2039.

IR (film) λₘₐₓ: 2927, 2861, 1731, 1662, 1127, 1077, 1051;

¹H NMR (600 MHz, CDCl₃): δ 5.85 (s, 1H), 4.71 (dd, J=29.0, 4.2 Hz, 2H), 4.23 (d, J=15.3 Hz, 1H), 4.17 (q, J=3.0 Hz, 1H), 4.10 (d, J=15.3 Hz, 1H), 3.96 (d, J=12.2 Hz, 1H), 3.57 (dd, J=12.2, 1.5 Hz, 1H), 2.96-2.88 (m, 1H), 2.68 (ddd, J=13.0, 4.9, 2.6 Hz, 1H), 2.54 (ddd, J=17.7, 14.8, 4.9 Hz, 1H), 2.44-2.31 (m, 5H), 2.21 (tdd, J=14.4, 4.5, 2.1 Hz, 1H), 2.09 (dd, J=13.6, 3.6 Hz, 1H), 2.07-2.02 (m, 1H), 1.92 (dq, J=11.9, 3.7 Hz, 1H), 1.86 (dd, J=13.5, 3.0 Hz, 1H), 1.82-1.70 (m, 3H), 1.56-1.52 (m, 1H), 1.47 (ddt, J=17.7, 10.8, 5.7 Hz, 1H), 1.15 (t, J=7.6 Hz, 3H), 1.10 (dt, J=11.2, 1.6 Hz, 1H), 0.94 (s, 3H);

¹³C NMR (CDCl₃, 151 MHz): δ 199.3, 197.6, 174.3, 166.3, 126.7, 94.9, 92.9, 73.4, 64.6, 56.9, 52.3, 47.8, 46.3, 43.6, 37.1, 33.9, 33.7, 32.6, 32.2, 31.4, 30.3, 27.9, 23.5, 17.5, 9.1.

### Example 1

### Compound 11

### (8S,9S,10S,11S,13S,14S,17R)-17-(2-chloroacetyl)-11-hydroxy-10-(hydroxymethyl-13-methyl-3-oxo-2,3,6,7,8,9,10,11,12,13,14,15,16,17-tetradecahydro-1H-cyclopenta[a]phenanthren-17-yl propionate

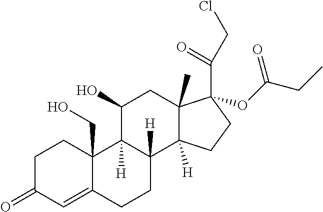

### Compound 1

Intermediate 9 (7.0 mg, 15 μmmol) was dissolved in THF (1.0 mL) and 6 M HCl (0.1 mL), and the solution was heated at 75° C. for 5 h under Ar. The reaction was then cooled to room temperature, and diluted with ethyl acetate (3 mL) and brine (3 mL). The mixture was partitioned in a separate funnel, and the aqueous phase was separated, and extracted with ethyl acetate (3 mL). The combined organic phases were dried over Na₂SO₄, and concentrated under vacuo. The resulting residue was purified by PTLC (DCM: MeOH=15:1) to afford compound 1 (4.1 mg, 60%)

Physical state: white foam;

R_{f}=0.20 (silica gel, 1:1 EtOAc:hexanes);

[α]^{D} ,25=+31.7° (c=0.24, CHCl₃);

HRMS (m/z): calcd for C₂₄H₃₄ClO₆, [M+H]⁺, 453.2038. found, 453.2052.

IR (film) λₘₐₓ: 2922, 2853, 1732, 1662, 1458, 1191, 1069, 670;

¹H NMR (400 MHz, CDCl₃): δ 5.78 (s, 1H), 4.47 (q, J=3.1 Hz, 1H), 4.30 (d, J=15.4 Hz, 1H), 4.13 (d, J=15.5 Hz, 1H), 4.12 (d, J=11.4 Hz, 1H), 3.60 (d, J=11.7 Hz, 1H), 2.94-2.86 (m, 1H), 2.60 (dd, J=13.1, 4.5 Hz, 1H), 2.56-2.45 (m, 1H), 2.42-2.26 (m, 5H), 2.16 (ddd, J=14.9, 12.6, 3.8 Hz, 2H), 2.08-1.99 (m, 1H), 1.89-1.75 (m, 3H), 1.75-1.64 (m, 2H), 1.49 (td, J=11.0, 5.7 Hz, 1H), 1.21 (dt, J=12.3, 4.4 Hz, 1H), 1.14 (t, J=7.5 Hz, 3H), 0.99 (s, 3H), 0.92-0.80 (m, 1H);

¹³C NMR (CDCl₃, 151 MHz): δ 199.4, 198.0, 174.3, 168.7, 124.0, 94.5, 67.2, 63.70, 56.6, 52.9, 47.0, 46.5, 44.2, 39.1, 34.0, 32.9, 32.3, 31.4, 31.2, 29.9, 27.8, 23.8, 17.5, 9.0.

### Intermediate 11

### (4aS,4a′S,8aS,9aS,10R,12aS,12bS,14R)-10-(2-chloroacetyl)-14-fluoro-10-hydroxy-9a-methyl-3,4,8a,9,9a, 10,11,12,12a,12b, 13,14-dodecahydro-5H-cyclopenta[1,2]phenanthro[4,4b-de][1,3]dioxepin-2(4a¹H)-one

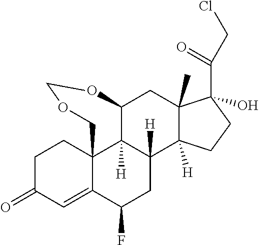

To a solution of intermediate 8 (200 mg, 0.489 mmol, 1.0 equiv) in Ac₂O (5 mL) was added NaI (293 mg, 1.956 mmol, 4.0 equiv), followed by TMSCl (0.25 mL, 1.956 mmol, 4.0 equiv) at 0° C. The resulting mixture was stirred at 0° C. for 1 h before being diluted with EtOAc (10 mL), and quenched with sat. aq. Na₂S₂O₃ (10 mL). The aqueous phase was extracted with EtOAc (2×10 mL), and the combined organic phase was washed sequentially with sat. aq. NaHCO₃ (10 mL) and brine (10 mL), dried over Na₂SO₄ and concentrated in vacuo. The resulting oil was used for the next step without further purification.

The resulting oil was dissolved in MeCN (5 mL) and cooled to 0° C. Selectfluor (225 mg, 0.635 mmol, 1.3 eq) was then added and the resulting mixture was stirred at 0° C. for 1 h. The reaction was quenched by the addition of H₂O (5 mL), and extracted with EtOAc (3×15 mL). The combined organic portions were washed with brine (10 mL), dried over Na₂SO₄ and concentrated in vacuo. The resulting oil was purified by column chromatography (silica gel, 1:3 EtOAc:hexanes) to afford intermediate 11 (102 mg, 49%).

Physical state: white solid;

R_{f}=0.44 (silica gel, 2:3 EtOAc:hexanes);

[α]^{D},25=+109.1° (c=0.11, CHCl₃);

HRMS (m/z): calcd for C₂₂H₂₈ClFO₅, [M+H]⁺, 427.1682. found, 427.1681.

IR (film) λₘₐₓ: 3484, 2945, 1728, 1685, 1666, 1461, 1369, 1230, 1130, 1083, 1052, 918;

¹H NMR (600 MHz, CDCl₃): δ 5.96 (d, J=5.4 Hz, 1H), 5.00 (d, J=48.8 Hz, 1H), 4.74 (d, J=4.2 Hz, 1H), 4.67 (d, J=4.2 Hz, 1H), 4.61 (d, J=16.4 Hz, 1H), 4.28 (d, J=16.4 Hz, 1H), 4.17 (s, 1H), 4.14 (dd, J=12.7, 3.5 Hz, 1H), 3.55 (d, J=12.7 Hz, 1H), 2.91-2.83 (m, 1H), 2.80-2.73 (m, 1H), 2.63 (ddd, J=19.2, 14.6, 4.9 Hz, 1H), 2.48-2.42 (m, 2H), 2.28 (ddt, J=13.1, 8.1, 3.8 Hz, 1H), 2.02 (dd, J=13.4, 3.0 Hz, 1H), 1.88-1.77 (m, 2H), 1.75 (dd, J=13.4, 3.0 Hz, 1H), 1.55-1.48 (m, 3H), 1.36 (dt, J=45.7, 13.7 Hz, 1H), 1.08 (d, J=11.0 Hz, 1H), 0.95 (s, 3H);

¹³C NMR (CDCl₃, 151 MHz): δ 203.0, 199.8, 176.7, 157.7, 130.7, 92.8, 90.8 (d, J=283.8 Hz), 73.1, 64.6, 55.8, 51.4, 48.7, 47.9, 42.8, 36.7, 35.4 (d, J=22.8 Hz), 34.8, 34.0, 32.4, 26.0, 23.2, 18.0;

¹⁹F NMR (376 MHz, CDCl₃): δ −169.1.

### Intermediate 12

### (4aS,4a¹S,8aS,9aS,10R,12aS,12bS,14R)-10-(2-chloroacetyl)-14-fluoro-9a-methyl-2-oxo-2,3,4,4a¹,8a,9,9a,10,11,12,12a,12b,13,14-tetradecahydro-5H-cyclopenta[1.2]phenanthro[4,4b-de][1,3]dioxepin-10-yl propionate

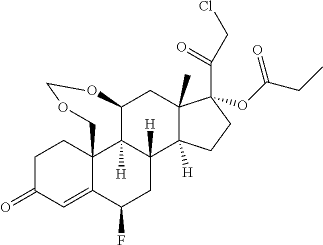

To a solution of intermediate 11 (18.0 mg, 42.2 μmol, 1.0 equiv) in 4:1 DCM:pyridine (1 mL) was added (EtCO)₂O (25 μL, 195.9 μmol, 4.6 equiv) and DMAP (5.2 mg, 42.2 μmol, 1.0 equiv). The resulting mixture was heated at 40° C. for 8 h, and then diluted with EtOAc (5 mL) and washed with H₂O (5 mL). The aqueous phase was extracted with EtOAc (2×2.5 mL) and the combined organic portions were washed with brine (5 mL), dried over Na₂SO₄ and concentrated in vacuo. The resulting oil was purified by PTLC (silica gel, 2:3 EtOAc:hexanes) to afford intermediate 12 (13.0 mg, 64%).

Physical state: white foam;

R_{f}=0.50 (silica gel, 2:3 EtOAc:hexanes);

[α]^{D},25=+49.4° (c=1.1, CHCl₃);

HRMS (m/z): calcd for C₂₅H₃₂ClFO₆, [M+H]⁺, 483.1944. found, 483.1941.

IR (film) λₘₐₓ: 2943, 1732, 1687, 1461, 1368, 1292, 1261, 1228, 1198, 1130, 1055, 919;

¹H NMR (600 MHz, CDCl₃): δ 5.97 (d, J=5.3 Hz, 1H), 5.01 (dd, J=48.8, 2.7 Hz, 1H), 4.74 (d, J=4.1 Hz, 1H), 4.67 (d, J=4.1 Hz, 1H), 4.25 (d, J=15.4 Hz, 1H), 4.18 (d, J=3.1 Hz, 1H), 4.14 (dd, J=12.7, 3.4 Hz, 1H), 4.12 (d, J=15.1 Hz, 1H), 3.56 (d, J=12.7 Hz, 1H), 2.97-2.91 (m, 1H), 2.82-2.73 (m, 1H), 2.64 (ddd, J=19.1, 14.9, 4.9 Hz, 1H), 2.54-2.42 (m, 2H), 2.41-2.33 (m, 2H), 2.28 (ddt, J=12.9, 8.0, 3.7 Hz, 1H), 2.09 (dd, J=13.4, 3.0 Hz, 1H), 1.88 (dd, J=13.4, 3.0 Hz, 1H), 1.82-1.73 (m, 3H), 1.36 (dt, J=44.5, 13.9 Hz, 1H), 1.14 (td, J=7.6, 1.2 Hz, 3H), 1.11-1.05 (m, 2H), 0.96 (s, 3H);

¹³C NMR (CDCl₃, 151 MHz): 5199.6, 197.5, 176.8, 174.2, 157.5, 130.7, 99.7, 93.8 (d, J=275.6 Hz), 72.9, 64.7, 55.9, 52.2, 47.9, 46.4, 42.8, 37.1, 35.3, 34.0, 32.5, 31.3, 27.9, 26.1, 23.4, 17.4, 9.1;

¹⁹F NMR (376 MHz, CDCl₃): δ −169.1.

### Example 2

### Compound 2

### (6R,8S,9S,10S,11S,13S,14S,17R)-17-(2-chloroacetyl)-6-fluoro-11-hydroxy-10-(hydroxymethyl)-13-methyl-3-oxo-2,3,6,7,8,9,10,11,12,13,14,15,16,17-tetradecahydro-1H-cyclopenta[a]phenanthren-17-yl propionate

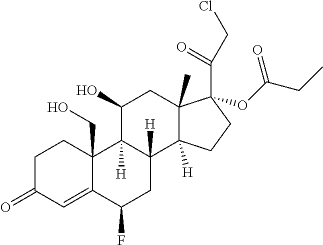

### Compound 2

Intermediate 12 (4.0 mg, 8.3 μmmol) was dissolved in THF (1 mL) and 6 M HCl (0.1 mL), and the solution was heated at 80° C. for 24 h under Ar (Alternatively, the deprotection could be conducted by heating the THF solution of intermediate 12 at 120° C. under microwave irradiation conditions for 15 min). The reaction was then cooled to room temperature, and diluted with ethyl acetate (3 mL) and brine (3 mL). The mixture was partitioned in a separate funnel, and the aqueous phase was separated, and extracted with ethyl acetate (3×3 mL). The combined organic phases were dried over Na₂SO₄, and concentrated under vacuo. The resulting residue was purified by PTLC (15:1 DCM:MeOH) to afford compound 2 (1.2 mg, 31%)

Physical state: white foam;

R_{f}=0.40 (silica gel, 15:1 DCM:MeOH, run twice);

[α]^{D},25=+27.0° (c=0.17, CHCl₃);

HRMS (m/z): calcd for C₂₄H₃₃ClFO₆, [M+H]⁺, 471.1944. found, 471.1951.

IR (film) λₘₐₓ: 2932, 1729, 1668, 1458, 1272, 1194, 1080, 732;

¹H NMR (600 MHz, CDCl₃): δ 6.15 (d, J=1.9 Hz, 1H), 5.30-5.15 (m, 1H), 4.55 (d, J=3.0 Hz, 1H), 4.32 (d, J=15.2 Hz, 1H), 4.15 (d, J=15.2 Hz, 1H), 3.98 (d, J=11.7 Hz, 1H), 3.83 (d, J=11.8 Hz, 1H), 2.93 (dd, J=13.5, 11.0 Hz, 1H), 2.61 (dd, J=13.3, 4.6 Hz, 1H), 2.53 (ddd, J=18.3, 13.6, 4.8 Hz, 1H), 2.48-2.35 (m, 5H), 2.32-2.25 (m, 1H), 2.23 (dd, J=14.3, 3.2 Hz, 1H), 1.91-1.76 (m, 6H), 1.44-1.34 (m, 1H), 1.17 (t, J=7.6 Hz, 3H), 1.03 (s, 3H).

¹³C NMR (CDCl₃, 151 MHz): δ 198.3, 197.8, 174.3, 164.3 (d, J=11.2 Hz), 119.9 (d, J=14.0 Hz), 94.2, 87.7 (d, J=185.7 Hz), 67.1, 65.0, 56.3, 52.9, 47.2, 46.4, 45.1 (d, J=2.1 Hz), 39.2, 38.2 (d, J=18.3 Hz), 33.8, 31.2, 31.1, 30.2 (d, J=11.5 Hz), 27.8, 23.8, 17.5, 9.0;

¹⁹F NMR (CDCl₃, 376 MHz): δ −181.9.

### Example 3

### Glucocorticoid Receptor (GR) Binding Assay

Glucocorticoid receptor binding was determined by competitive receptor binding assay using the PolarScreen™ Glucocorticoid Receptor Competitor Assay Kit, Green (Life Technologies, catalogue number A15897) in a 384-well format according to the manufacturer's instructions. Briefly, serial dilutions of test and reference compounds were prepared in 384-well plates. Subsequently, the steroidal competitor Fluormone™ GS1 and recombinant glucocorticoid receptor (GR) were added to all wells and the plate was incubated for 2 hours at room temperature and protected from light. Finally, polarization values in each well were determined by reading the plate in an EnVision (PerkinElmer) device.

### Example 4

### Mineralocorticoid Receptor (MR) Agonism and Antagonism Assay

Agonistic and antagonistic interaction with the mineralocorticoid receptor was determined using the GeneBLAzer® MR DA Assay Kit (Life Technologies, catalogue number K1409) in a 96-well format according to the manufacturer's instructions. For determination of MR agonistic effect, cells were incubated with 0.14 nM-1 uM of test compound; the MR agonist aldosterone was included as a positive control in the same concentration range. After 16 h incubation, assay substrate (LiveBLAzer™-FRET B/G) was added to all wells and cells were incubated for 2 h followed by fluorometric detection (409/447 nm excitation and emission wavelength, respectively) of substrate conversion by beta-lactamase.

For determination of MR antagonistic activity, cells were incubated with 0.14 nM-1 uM of test compound; the MR antagonist spironolactone was included as a positive control in the same concentration range. After 0.5 h, 10 nM of the MR agonist aldosterone was added to all wells and cells were incubated for another 15.5 h. After that, assay substrate (LiveBLAzer™-FRET B/G) was added to all wells and cells were incubated for 2 h followed by fluorometric detection (409/447 nm excitation and emission wavelength, respectively) substrate conversion by beta lactamase.

### Example 5

### Interleukin-12B (IL-12B) Release from Primary Human Monocytes (PBMC)

The assay was adopted from a previously reported method (Schäcke et al, Br 3 Pharmacol (2009); 158(4): 1088-103) and is based on PBMC isolated from human blood. Stimulation of these cells with lipopolysaccharide (LPS) induces—among many other processes—the synthesis and secretion of the cytokine IL-12B. Both steroidal (glucocorticoids) and non-steroidal glucocorticoid receptor agonists act anti-inflammatory—e.g. by preventing DNA-binding of inflammatory transcription factors like AP-1 or NFkB—resulting in decreased synthesis/secretion of IL12B.

For compound testing, frozen stocks of PBMC were thawed, seeded in 96-well plates (1.2E5 cells/well) and then incubated in RPMI-1640 culture medium with 100 ng/ml LPS for 18 h with or without test compounds (pre-incubation with reference/test compounds for 1 h before addition of LPS). Then, IL-12B concentration in the cell culture supernatants was detected by hIL-12B-specific ELISA (Quantikine® human IL-12 p40 Immunoassay, R&D Systems Cat #SP400).

Compounds of the present invention were tested in the above in vitro assays. Relative IC₅₀ values are displayed in Table 1

TABLE 1

| | | Mineral corticoid | |
|---|---|---|---|
| | | receptor (MR) | Interleukin-12B |
| | GR binding; | antagonism; | (IL-12B); Rel |
| | Rel IC₅₀ (nM) | Rel IC₅₀ (nM) | IC₅₀ (nM) |
| Compound 1 | 25 | >1000 | 36 |
| Compound 2 | 40 | n.d. | 31 |
| Reference: | 7 | 137 | <1 |
| Bethamethasone | | | |
| valerate | | | |
| Clobetasol | 6 | 171 | <1 |
| propionate | | | |
| Spironolactone | n.d | 58 | n.d. |
| (MR antagonist) | | | |

1. A compound according to formula I 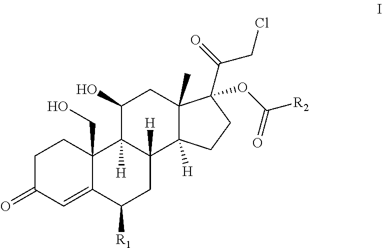
wherein
R₁ is selected from the group consisting of hydrogen and fluoro;
R₂ is selected from C₁-C₄ alkyl;
and hydrates or solvates thereof.

2. The compound according to claim 1, wherein R₁ is hydrogen.

3. The compound according to claim 1, wherein R₁ is fluoro.

4. The compound according to claim 1 wherein R₂ is ethyl.

5. A compound according to claim 1 selected from the list of
(8S,9S,10S,11S,13S,14S,17R)-17-(2-chloroacetyl)-11-hydroxy-10-(hydroxymethyl)-13-methyl-3-oxo-2,3,6,7,8,9,10,11,12,13,14,15,16,17-tetradecahydro-1H-cyclopenta[a]phenanthren-17-yl propionate (Compound 1) and
(6R,8S,9S,10S,11 S,13S,14S,17R)-17-(2-chloroacetyl)-6-fluoro-1-hydroxy-10-(hydroxymethyl)-13-methyl-3-oxo-2,3,6,7,8,9,10,11,12,13,14,15,16,17-tetradecahydro-1H-cyclopenta[a]phenanthren-17-yl propionate (Compound 2) and hydrates or solvates thereof.

6. A compound according to claim 1 for use in therapy.

7. A compound according to claim 1 for use in treatment of inflammatory diseases.

8. The compound according to claim 7 wherein the inflammatory disease is selected from the group consisting of psoriasis, eczema and atopic dermatitis.

9. A pharmaceutical composition comprising a compound according to claim 1 together with a pharmaceutically acceptable vehicle or excipient or pharmaceutically acceptable carrier(s).

10. The pharmaceutical composition according to claim 9 together with one or more other therapeutically active compound(s).

11. The use of a compound according to claim 1 in the manufacture of a medicament for the prophylaxis, treatment or amelioration of inflammatory diseases.

12. The use according to claim 11, wherein the inflammatory disease is selected from the group consisting of psoriasis, eczema and atopic dermatitis.

13. A method of preventing, treating or ameliorating inflammatory diseases, the method comprising administering to a person suffering from at least one of said diseases an effective amount of one or more compounds according to claim 1, optionally together with a pharmaceutically acceptable carrier or one or more excipients, optionally in combination with other therapeutically active compounds.

14. A method according to claim 13, wherein the inflammatory disease is selected from the group consisting of psoriasis, eczema and atopic dermatitis.

15. A compound according to general formula II 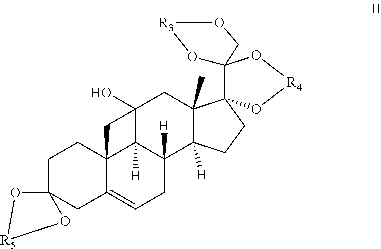
Wherein R₃ and R₄ independently represent —CH₂—, —CH(CH₃)— or —C(CH₃)₂— and wherein
R₅ represents —(CH₂)₂—, —(CH₂)₃— or —CH₂—C(CH3)₂-CH₂—.

16. The compound according to claim 15 which is
(6a′S,6a1′S,8a′S,9′R,11a′S, 11b′S)-8a′-methyl-1′,3′,5′,6′,8′,8a′, 10′,11′,11a′, 11b′-decahydro-7′H-trispiro[[1,3]dioxolane-2,4′-cyclobuta[de]cyclopenta[a]phenanthrene-9′,4″-[1,3]dioxolane-5″,4′″-[1,3]dioxolan]-7a′(6a1′H)-ol.

17. A compound according to general formula III 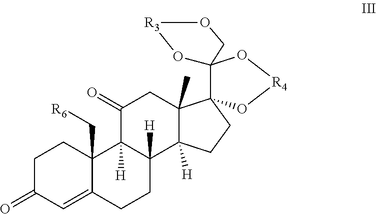
wherein R₃ and R₄ independently represent —CH₂—, —CH(CH₃)— or —C(CH₃)₂— and
wherein R₆ represents halogen or hydroxyl.

18. The compound according to claim 17 which is
(8S,9S,10 S,13S,14S,17R)-10-(hydroxymethyl)-13-methyl-1,2,6,7,8,9,10,12,13,14,15,16-dodecahydrodispiro[cyclopenta[α]phenanthrene-17,4′-[1,3]dioxolane-5′,4″-[1,3]dioxolane]-3,11-dione.

19. The compound according to claim 2 wherein R₂ is ethyl.

20. The compound according to claim 3 wherein R₂ is ethyl.
